(19) United States
(12) Patent Application Publication
(10) Pub. No.: US 2009/0211576 A1
(43) Pub. Date: Aug. 27, 2009

(54) SAFETY AND ABUSE DETERRENT IMPROVED DEVICE

(76) Inventors: Timo Lehtonen, Turku (FI); Matthew Young, Over (GB); Joerg Grosser, Lappersdorf (DE); Juergen Ernst Pfrang, Kallmuenz (DE); Udo Leuschner, Renensburg (DE)

Correspondence Address: MEYERTONS, HOOD, KIVLIN, KOWERT & GOETZEL, P.C., P.O. BOX 398, AUSTIN, TX, 78767-0398, US

(21) Appl. No.: 12/244,617

(22) Filed: Oct. 2, 2008

Related U.S. Application Data
(60) Provisional application No. 60/977,016, filed on Oct. 2, 2007.

(51) Int. Cl. A61M 15/00

(52) U.S. Cl. 128/203.12

(57) ABSTRACT

Modifications of an inhaler and method of use thereof are described. In some embodiments, an inhaler may include one or more systems and/or methods that function to inhibit a child from accessing medicant from an inhaler. A system may function to inhibit a child from accessing an opening of an inhaler from which medicants are dispensed. In some embodiments, a system may inhibit a child from removing a cap for an inhaler. A system may function to inhibit a child from actuating an activation mechanism for an inhaler. A system may function to inhibit a user and/or a child from accidentally actuating an activation mechanism for an inhaler multiple times possibly resulting in an overdose. A system may function to inhibit an activation mechanism for an inhaler from actuating after the activation mechanism has been actuated a predetermined number of times. An inhaler may include a security fitting which functions to permanently plug an inhaler once a prescribed user is finished with the inhaler.

### PRIORITY CLAIM

This application claims priority to U.S. Provisional Application Ser. No. 60/977,016 Entitled: “SAFETY AND ABUSE DETERRENT IMPROVED DEVICE” to Lehtonen et al., filed on Oct. 2, 2007.

### BACKGROUND OF THE INVENTION

1. Field of the Invention

The present disclosure generally relates to systems and methods directed towards increasing the safety of multiple dose inhalers. More particularly, the disclosure generally relates to systems and methods for inhibiting the inadvertent and/or intentional abuse of a medicant dispensed from a multiple dose inhaler.

2. Description of the Relevant Art

Persistent pain such as pain related to cancer can have a severe impact on a patient's quality of life, often interfering with the ability to eat, sleep, and interact with others. Patients in pain may be less willing to undergo treatment, less able to cope with side effects, and sometimes less willing to live. Control of pain is therefore an important and essential component of therapy, especially of cancer therapy. Mild pain can be treated with analgesic agents such as acetaminophen, NSAIDs and codeine, while persisting or increasing pain can be treated with weak or strong opioids. Moderate to severe chronic cancer pain can be treated with opioids where the therapeutic goal is to achieve maximal analgesia and minimize the occurrence of side effects, especially of adverse effects. While morphine is recommended as the opioid of first choice for the treatment of moderate to severe cancer pain, this recommendation is based largely on familiarity with morphine use and its availability and cost, not on proven clinical superiority over other opioids, which could provide significant and rapid analgesia at lower doses and without significant side effects if available in appropriate formulations and suitably administered. Alternative opioids and alternative routes of administration are needed to optimize the balance between analgesia and adverse events for individual patients. Tolerance build-up and the consequence of wide window of titration, with the titration not always successful, are problems commonly associated with opioid treatments. For instance, current transmucosal fentanyl treatments require a titration range of up to 8-fold of the starting dose to achieve successful analgesia in up to approximately 70% of the patients treated.

Drug formulations for the oral delivery of pharmaceuticals have been used for centuries. More recently, numerous compositions and methods have been developed for the controlled release of pharmaceuticals. Such controlled-release systems and methods can be useful for many reasons. One reason is that controlled-release delivery systems can limit the number of doses a patient must take over a period of time thus improving compliance with a dosing regimen. Controlled release of pharmaceuticals is particularly critical with drugs that are habit forming, as the controlled release of the medication can significantly reduce the likelihood of a patient developing an addiction to the substance.

Abuse of narcotic substances is particularly problematic. Such drugs are highly habit forming when misused and thus are in high demand by drug abusers. In contrast, there are numerous legitimate users of narcotic substances for the treatment of extreme pain.

In addition to medicant abuse by addicts, systems may function to inhibit access to medicants by underage children without proper supervision and/or inhibit accidental overdosing by prescribed users.

Therefore there remains a significant need in the art for multiple dose inhaler forms that are resistant to inadvertent overdosing and/or attempts by potential abusers to bypass the controlled release characteristics of conventional multiple dose inhalers.

### SUMMARY

The present invention solves the problems described above by providing systems and methods for inhibiting the inadvertent and/or intentional abuse of a medicant dispensed from a multiple dose inhaler for pulmonary inhalation administration.

In some embodiments, an inhaler may include an inhaler body and a cap. An inhaler body may include a dispenser opening and two or more recesses. A dispenser opening may be positioned at a proximal end of the inhaler body. A dispenser opening may function to dispense a dose of medicant upon activation of the inhaler body. Two or more recesses may be positioned on opposing sides of an exterior surface of the proximal end of the inhaler body. A cap may include two or more tabs. Two or more of the tabs may be positionable in two or more of the recesses such that when the tabs are positioned in the recesses the cap is coupled to the proximal end of the inhaler body. When the tabs are positioned in the recesses children are inhibited from accessing the dispenser opening.

In some embodiments, an inhaler may include an inhaler body and a dispensing lock. An inhaler body may include a first portion and a second portion. A first portion may include a dispenser opening. A dispenser opening may function to dispense a dose of medicant upon activation of the inhaler body. An inhaler body may be activated by rotation of the first portion relative to the second portion. A dispensing lock may function to inhibit rotation of the first portion relative to the second portion of the inhaler when the dispensing lock is in a first locked position. A dispensing lock may function to allow rotation of the first portion relative to the second portion of the inhaler when the dispensing lock is in a second unlocked position.

In some embodiments, an inhaler may include an inhaler body, a cap, and a multiple dose inhibitor system. An inhaler body may include a dispenser opening. A dispenser opening may be positioned at a proximal end of the inhaler body. A dispenser opening may function to dispense a dose of medicant upon activation of the inhaler body. A cap may function to couple to the proximal end of the inhaler body. A cap may function to inhibit access to the dispenser opening. A multiple dose inhibitor system may function to inhibit activation of the inhaler body when the cap is uncoupled to the inhaler body.

In some embodiments, an inhaler may include an inhaler body and a dose limitation system. An inhaler body may include a dispenser opening. A dispenser opening may be positioned at a proximal end of the inhaler body. A dispenser opening may function to dispense a dose of medicant upon activation of the inhaler body. A dose limitation system may function to inhibit activation of the inhaler body after the inhaler body has been activated a predetermined number of times.

In some embodiments, an inhaler may include an inhaler body and a security fitting. An inhaler body may include a dispenser opening. A dispenser opening may be positioned at a proximal end of the inhaler body. A dispenser opening may function to dispense a dose of medicant upon activation of the inhaler body. A security fitting may be positionable in the dispenser opening. A security fitting may function to inhibit access to medicant positioned in the inhaler body when the security fitting is positioned in the dispenser opening.

### BRIEF DESCRIPTION OF THE DRAWINGS

Advantages of the present invention may become apparent to those skilled in the art with the benefit of the following detailed description of the preferred embodiments and upon reference to the accompanying drawings briefly described herein below.

FIG. 1 depicts a representation of a perspective view of an embodiment of an inhaler cap.

FIG. 2 depicts a representation of a perspective view of an embodiment of an inhaler body.

FIG. 3 depicts a representation of a perspective view of an embodiment of an assembled inhaler body and cap.

FIG. 4 depicts a representation of a perspective view of an embodiment of an inhaler body.

FIG. 5 depicts a representation of a perspective view of an embodiment of an inhaler reservoir.

FIGS. 6A-F depict a representation of a perspective view of an embodiment of an inhaler including a dispensing lock and an embodiment of a method using the inhaler.

FIGS. 7A-D depict a representation of a perspective view of an embodiment of an interior portion of an inhaler including a dispensing lock and an embodiment of a method using the inhaler.

FIGS. 8A-D depicts a representation of a perspective view of an embodiment of a several parts used to form dispensing lock release mechanism of an inhaler.

FIG. 9 depicts a representation of a perspective view of an embodiment of an inhaler cap.

FIG. 10 depicts a representation of a perspective view of an embodiment of a cam for an inhaler.

FIG. 11A-C depict a representation of an embodiment of a multiple dose inhibitor system for an inhaler.

FIG. 12A-C depict a representation of a partial cross-sectional view of an embodiment of a multiple dose inhibitor system for an inhaler.

FIG. 13A-C depict a representation of a partial cross-sectional view of an embodiment of a multiple dose inhibitor system for an inhaler.

FIG. 14 depicts a representation of a perspective view of an embodiment of a locking pin assembly for an inhaler.

FIG. 15 depicts a representation of a perspective view of an embodiment of a cap for an inhaler.

FIG. 16 depicts a representation of a perspective view of an embodiment of a second portion cover for an inhaler.

FIG. 17 depicts a representation of a top perspective view of an embodiment of an inhaler with a cap removed.

FIGS. 18A-B depict a representation of a perspective view of an embodiment of a reservoir for an inhaler.

FIGS. 19A-C depict a representation of a perspective view of an embodiment of a controlled dispenser system for an inhaler.

FIGS. 20A-B depict a representation of a perspective and a cross-sectional view of an embodiment of a second portion cover for an inhaler.

FIGS. 21A-C depict a representation of a perspective and a cross-sectional view of an embodiment of a reservoir for an inhaler.

FIGS. 22A-B depict a representation of a perspective view of an embodiment of a counter system for an inhaler.

FIG. 23 depicts a representation of a perspective view of an embodiment of a cam for an inhaler.

FIGS. 24A-B depict a representation of a perspective view of an embodiment of a spring for an inhaler.

FIGS. 25A-B depict a representation of a partial cross-sectional view of an embodiment of a second portion cover with a spring and a counter for an inhaler.

FIGS. 26A-B depict a representation of a cross-sectional view of an embodiment of a spring and a counter for an inhaler.

FIGS. 27A-C depict a representation of a cross-sectional view of an embodiment of a spring and stop for an inhaler.

FIGS. 28A-B depict a representation of a cross-sectional view of an embodiment of a ratchet system for an inhaler.

FIG. 29 depicts a representation of an embodiment of a security fitting for an inhaler.

FIG. 30 depicts a representation of a cross-sectional view of an embodiment of a security fitting for an inhaler.

FIG. 31 depicts a representation of a perspective view of an embodiment of a security fitting for an inhaler.

FIG. 32 depicts a representation of a perspective view of an embodiment of a distal end of a security fitting for an inhaler.

FIG. 33 depicts a representation of an embodiment of a security fitting positioned in an inhaler.

FIG. 34 depicts a representation of a top view of an embodiment of a security fitting positioned in an inhaler.

FIG. 35 depicts a representation of a cross-sectional view of an embodiment of a security fitting positioned in an inhaler.

FIG. 36 depicts a representation of a cross-sectional view of an embodiment of a proximal end of a security fitting positioned in an inhaler.

FIG. 37 depicts a representation of a cross-sectional view across frangible area of an embodiment of a security fitting.

FIG. 38 depicts a representation of an embodiment of a seal recess of a security fitting.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

Unless defined otherwise, all technical and scientific terms used herein have the same meaning as commonly understood by one of ordinary skill in the art.

The term “cam” as used herein generally refers to a portion of an inhaler mounted on a rotating shaft, used to produce variable or reciprocating motion in another engaged or contacted part.

The term “connected” as used herein generally refers to pieces that may be joined or linked together.

The term “coupled” as used herein generally refers to pieces that may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

The term “distal” as used herein generally refers to a point positioned furthest to a point of reference. The point of reference in the herein described application may be a user (e.g., a physician) of an embodiment of, for example, a pressurized syringe as described herein.

The term “fluids” as used herein generally refers to material that may be mechanically pumped under pressure. Fluids may include liquids or gases. In some embodiments, fluids may include air or liquids found in the body (e.g., saliva).

The term “proximal” as used herein generally refers to a point positioned nearest to a point of reference. The point of reference in the herein described application may be a user (e.g., a physician) of an embodiment of, for example, a pressurized syringe as described herein.

In addition to medicant abuse by addicts, systems and methods described herein at least may function to inhibit access to medicants by underage children without proper supervision and/or inhibit accidental overdosing by prescribed users and/or inhibit abuse by unprescribed users.

In some embodiments, an inhaler may include a system that functions to inhibit an unprescribed user from removing a cap from the inhaler. A cap may function to inhibit access and/or protect an opening from which medicants stored in an inhaler may be accessed. Caps for inhalers are common and known in the industry. In some embodiments, a cap may include a locking system that inhibits children from accessing a medicant without assistance.

In some embodiments, a child resistant inhaler cap may be used in combination with a TAIFUN® inhaler. Embodiments of a TAIFUN® inhalation device are described in U.S. Pat. Nos. 6,210,394, and 5,476,093, which are incorporated by reference as if fully set forth herein. Inhalers (e.g., TAIFUN®) may include approximately 1.5 doses of medicant retained in the mouthpiece after all of the prescribed doses have been used. It is not clear how much drug is available on the external surfaces of the mouthpiece (or internal surfaces of the cap) such that a child could access it by licking the inhaler, or sucking his/her fingers after touching it.

Current ‘ADD’ inhalers typically expel less than 5% extra medicant per extra actuation (e.g., if the reservoir is rotated 4 times, 115% of dose or less is likely). Therefore if a child rotates the reservoir several times before a prescribed patient inhales, the outcome is unlikely to involve a serious injury or fatality.

In some embodiments, an inhaler cap may inhibit a child from accessing any medicants by retaining the cap on the inhaler. In some embodiments, an inhaler cap may function to inhibit actuation of an inhaler. A system that functions to retain a cap on an inhaler may prevent a child from accessing ‘any’ drug. The system may not necessarily prevent a child from rotating the reservoir and actuating the inhaler. Systems may be easy to implement, and have a low impact on other aspects of the inhaler mechanism and function.

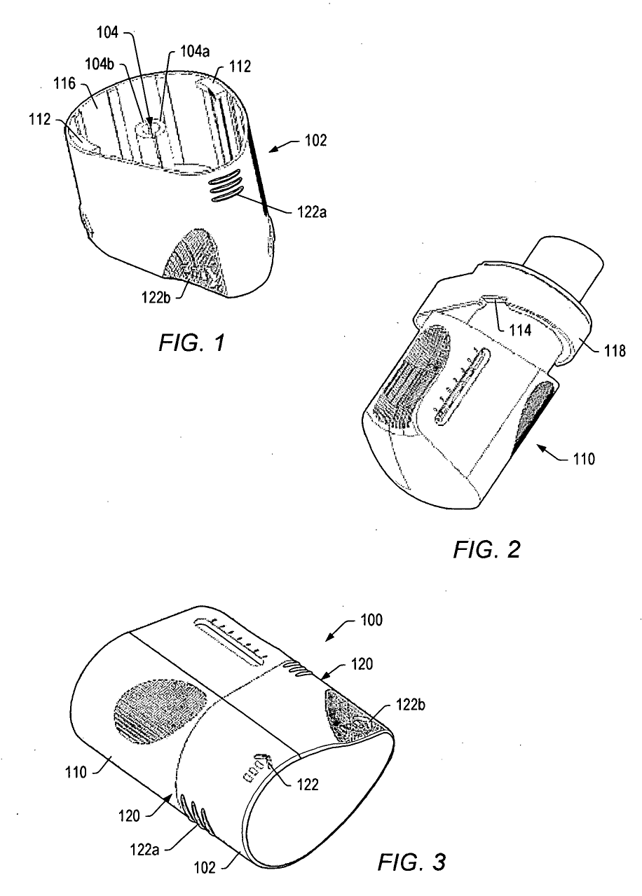

FIGS. 1-3 depict representations of perspective views of portions of inhaler 100. FIG. 1 depicts a representation of a perspective view of an embodiment of inhaler cap 102 for inhaler 100. FIG. 2 depicts a representation of a perspective view of an embodiment of inhaler body 110.

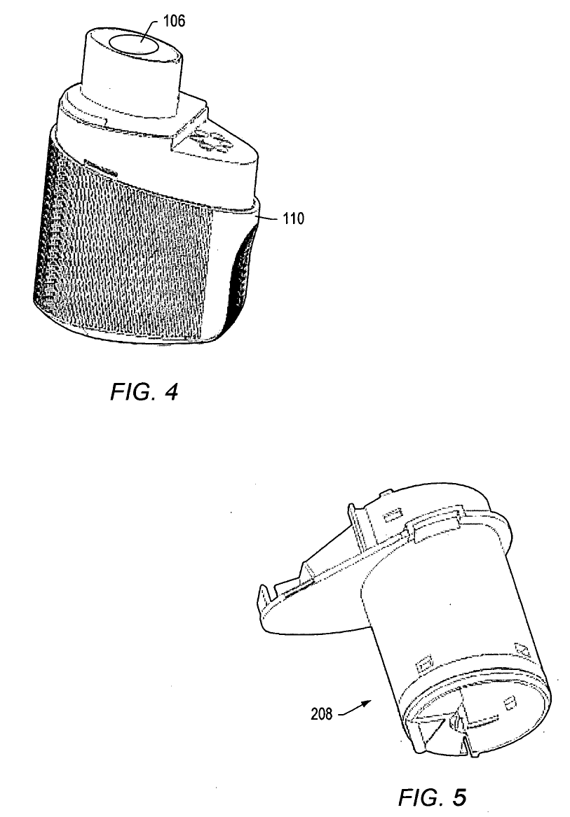

Inhaler cap 102 may include elongated member 104. Elongated member 104 may be positionable in medicant dispenser opening 106 of inhaler 100 (See FIG. 4). In some embodiments, elongated member 104 may form a friction fit coupling when positioned in dispenser opening 106. The friction fit coupling may function to assist in keeping a cap coupled to inhaler body 110.

In some embodiments, elongated member 104 may be formed by two or more portions. For example, elongated member 104 may be formed of two pieces 104a and 104b. Elongated member 104 may be formed from any material known to one skilled in the art. In some embodiments, elongated member 104 may be formed from a polymer, which upon being deflected and/or deformed within at least certain limits will return back to its original shape.

In some embodiments, inhaler 100 may include a locking system to inhibit children from removing cap 102 from inhaler body 110. A locking system may include one or more tabs 112. In some embodiments, a locking system may include two or more tabs 112. In some embodiments, a locking system may include three or more tabs 112. In some embodiments, a locking system may include four or more tabs 112.

In some embodiments, a locking system may include two tabs 112 positioned on an inside surface of cap 102. Tabs 112 may extend outward from the interior surface of the cap 102. Tabs 112 may extend from the interior surface towards the center of the cap within the plane of the tabs. Tabs 112 may take any appropriate shape. In some embodiments, tabs 112 may be formed as integral part of the cap (e.g., the cap and tabs may be formed as a single piece during manufacture). Tabs 112 may be positioned on substantially opposing interior surfaces of cap 102 (as depicted in FIG. 1).

In some embodiments, tabs 112 may be positionable in recesses 114 on inhaler body 110. Upon properly positioning tabs 112 in recesses 114 a child may be inhibited from removing cap 102 and accessing any medicant within the inhaler. Tabs 112 may have a complementary shape to recesses 114. Tabs 112 may have a substantially similar size to recesses 114; however tabs 112 may be sized slightly smaller relative to recesses 114 such that tabs 112 may fit in recesses 114.

In some embodiments, open end 116 of cap 102 may include a complementary size and shape to at least a portion 118 of second portion cover 212. Open end 116 may be configured to have a complementary shape to portion 118; however open end 116 may have a slightly larger size such that cap 102 may slide over portion 118. In some embodiments, cap 102 may be formed from any material known to one skilled in the art. In some embodiments, cap 102 may be formed from a polymer, which upon being deflected and/or deformed within certain limits will return back to its original shape. This may allow the cap to deform slightly such that tabs 112 may fit over portion 118 until the tabs are positioned in recesses 114.

FIG. 3 depicts a representation of a perspective view of an embodiment of an assembled inhaler body 110 and cap 102. In some embodiments, once positioned, cap 102 may be removed from inhaler body 110 by a user applying pressure to opposing sides 120 of the cap (as depicted in FIG. 3). The user may apply pressure to opposing sides 120 substantially simultaneously. Opposing sides 120 may be positioned substantially between the sides of cap 102 wherein tabs 112 are positioned. By applying pressure to opposing sides 120, the cap may be compressed inwards along an imaginary plane connecting opposing sides 120, such that to compensate for this distortion of opening 116 the sides of cap 102 wherein tabs 112 are positioned may expand outward along an imaginary plane connecting tabs 112. By extending tabs 112 outward away and out of recesses 114, cap 102 may be removed from inhaler body 110. These are operations, which a small child may find difficulty in performing, thus inhibiting children from removing the cap and accessing any medicants. Typically small children do not understand performing multiple actions at one time.

In some embodiments, cap 102 may include indicia 122, 122a, and 122b printed and/or formed into the surface of the cap. In some embodiments, indicia 122a may function to indicate to a user how to remove cap 102 from inhaler body 110. In some embodiments, indicia 122b may function to indicate to a user how to actuate inhaler 100. In some embodiments, indicia may be formed and/or etched into an outer surface of cap 102. The indicia may have the same color as that of the cap. This may result in several advantages. One advantage that indicia etched into the surface of a cap are less likely to wear away or rub off after extended handling and use by a user. A second advantage may include that the indicia are less obvious to a child and it is therefore less likely that the child will determine how to remove a cap from an inhaler body.

In some embodiments, an inhaler may include a system that functions to inhibit a child from activating an inhaler such that medicant is released. In some embodiments, such a dispensing lock may be used in combination with a child resistant cap as described herein or any other system described herein. A cap may function to inhibit access and/or protect an opening from which medicants stored in an inhaler may be accessed. In some embodiments, a dispensing lock may include a locking system that inhibits children from activating an inhaler and/or otherwise accessing a medicant within the inhaler without assistance.

A dispensing lock may be configured to inhibit children from activating an inhaler, while not substantially inhibiting prescribed users (e.g., including seniors/end-stage cancer sufferers who may have limited dexterity and vision) from activating the inhaler.

In some embodiments, a dispensing lock may be configured to require a user to perform at least two separate actions to be carried out by the user in order to access any medicant in an inhaler. In some embodiments, a dispensing lock may require that two separate actions be carried out substantially simultaneously by a user.

In some embodiments, a dispensing lock may be configured to comply with various government regulations known to one skilled in the art (e.g., ISO 8317:2003, which specifies both safety performance with children less than 52 months old and ease of use by Seniors).

FIGS. 4-9 depict representations of perspective views of embodiments of inhaler 100 including a dispensing lock 200 and an embodiment of a method using the inhaler. In some embodiments, inhaler 100 may include an activation mechanism. An activation mechanism may include any mechanism that prepares a predetermined dose of medicant for dispensing (e.g., inhalation). Preparing a dose of medicant for dispensing may include loading the dose of medicant in an air path of an inhaler.

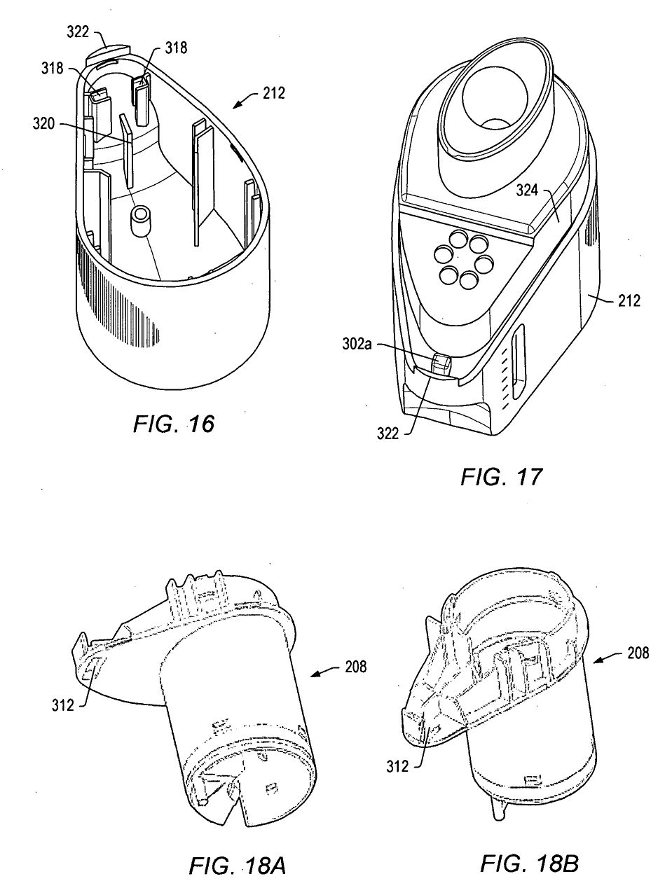

In some embodiments, the activation mechanism may require a user to rotate at least first portion 204 of an inhaler relative to at least second portion 206 of the inhaler about an axis. FIG. 4 depicts a representation of a perspective view of an embodiment of an inhaler body including medicant dispenser opening 106 and inhaler body 110. FIG. 5 depicts a representation of a perspective view of an embodiment of an inhaler reservoir 208 that is positioned in cover 212 (See FIG. 16). The activation mechanism may require a user to rotate at least first portion 204 of an inhaler relative to at least second portion 206 of the inhaler about an axis at least 180°. In some embodiments, an activation mechanism may require a user to rotate at least first portion 204 of an inhaler relative to at least second portion 206 of the inhaler about an axis at least 45°, at least 90°, at least 120°, at least 135°, at least 180°, at least 225°, at least 270°, at least 315°, or at least 360°.

In some embodiments, an inhaler may include dispensing lock 200. Dispensing lock 200 may function to inhibit the activation mechanism from functioning. When a dispensing lock is released an activation mechanism may function as intended. In some embodiments, a user may have to actively release a dispensing lock during at least actuation of an activating mechanism.

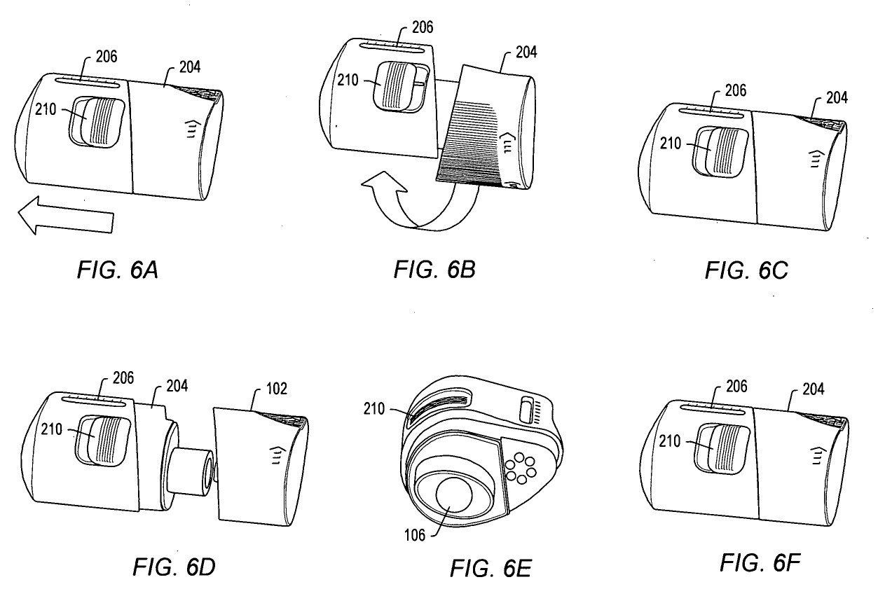

FIGS. 6A-F depict a representation of a perspective view of an embodiment of inhaler 100 including dispensing lock 200 and an embodiment of a method using the inhaler. Dispensing lock 200 may include release 210. In some embodiments, a release may include a sliding button or a sliding catch. In some embodiments, a release may include a push button. In some embodiments, a release may include a lever or a switch. In some embodiments, a release may be configured to move from a first position to a second position when activated by a user. The release may be configured to move back from the second position to the first position without assistance from the user as soon as the user disengages the release. For example, the release may include a spring mechanism. In some embodiments, a release may be configured to remain in a second position when moved from a first position until a user actuates the release. The design of a release may be intended to make it look innocuous to children, but comprehensible to adults for safety reasons.

In some embodiments, release 210 may remain in the unlocked position by a grip force of a user, so that they do not need to exert a continuous force to pull the sliding button back into the unlocked position whilst achieving a minimum required rotation. However if the user releases their grip during rotation the inhaler may revert to the locked condition. This functionality is intended to make the inhaler easier to use by patients.

FIG. 6A depicts an embodiment of inhaler 100 including first portion 204, second portion 206, and release 210. Release 210 is depicted in a locked or active position in FIG. 6A. When release 210 is in a locked or active position, the activation mechanism may be inhibited from functioning. FIG. 6B depicts inhalation system 100 with release 210 in an unlocked or inactive position. When release 210 is in a locked or inactive position, the activation mechanism may function normally. In the embodiment depicted in FIG. 6B activation system 202 may include a rotation mechanism wherein first portion 204 of an inhaler may be rotated relative to second portion 206 of the inhaler about an axis in order to prepare a dose of medicant to dispense. FIG. 6B depicts inhalation system 100 with release 210 in an unlocked position such that first portion 204 of an inhaler may be rotated relative to second portion 206 (e.g., 180° as depicted in FIG. 6B). FIG. 6C depicts inhalation system 100 with release 210 in a locked position after first portion 204 of an inhaler has been rotated relative to second portion 206 a full 360°, such that a predetermined single dose of medicant is prepared for dispensing.

In some embodiments, after activating an inhaler by rotating a first portion relative to a second portion, a user may remove cap 102 as depicted in FIG. 6D. After removing cap 102 a user may inhale the dose of medicant from dispenser opening 106 as depicted in FIG. 6E. After inhaling the dose of medicant from dispenser opening 106 a user may replace cap 102 with release 210 in a locked position and the inhaler in an unloaded state as depicted in FIG. 6F.

In some embodiments, release 210 must be unlocked before an inhaler may be activated such that a predetermined dose of medicant is loaded into the airpath ready for inhalation. In some embodiments, a user may be required to keep a release in the “unlocked position” over at least the first 90° of rotation of a first portion relative to a second portion of an inhaler before the user can complete the rotation and access a dose of medicant. In some embodiments, a user may be required to keep a release in the ‘unlocked position’ over at least the first 45°, over at least the first 90°, over at least the first 135°, over at least the first 180°, over at least the first 225°, over at least the first 270°, over at least the first 315°, or over at least the first 360° of rotation of a first portion relative to a second portion of an inhaler before the user can complete the rotation and access a dose of medicant.

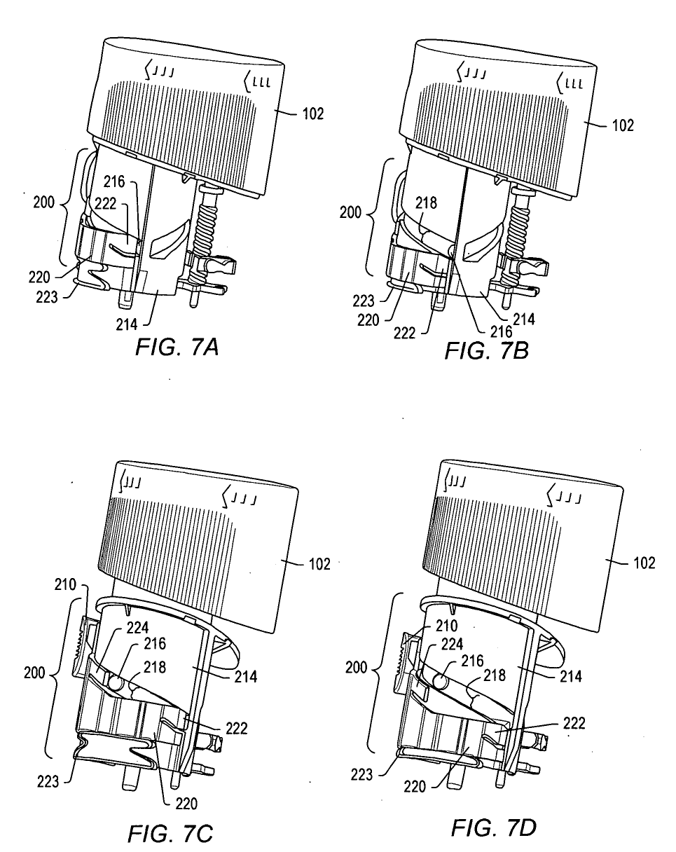

In some embodiments, a dispensing lock may include at least one locking stage. In some embodiments, a dispensing lock may include at least two separate “locking stages”. FIGS. 7A-D depict a representation of a perspective view of an embodiment of an interior portion of inhaler 100 including dispensing lock 200 and an embodiment of a method using the inhaler. FIGS. 7A-D depict a representation of a perspective view of an embodiment of inhaler 100 with second portion cover 212 removed from second portion 206 such that the mechanism may be more easily viewed for purposes of describing the mechanism.

In some embodiments, activating mechanism may include cam 214. Cam 214 may form a part of second portion 206. Typically second portion cover 212 may be coupled to cam 214. Reservoir 208 may be positioned in cam 214. Reservoir 208 may include cam tracker 216 coupled to the reservoir. Cam tracker 216 may include a raised portion that is formed as part of reservoir 208. When assembled, cam tracker 216 may extend out into cam track 218. Cam track 218 may include a channel or opening that extends through cam 214. Cam track 218 may extend around the circumference of cam 214. Extending cam track 218 around the circumference of cam 214 may allow reservoir 208 to rotate around an axis relative to cam 214 at least 360°.

In some embodiments, dispensing lock 200 may include lock arm 220. Lock arm 220 may be coupled to release 210. Lock arm 220 may include first engager 222 coupled to or formed as part of the lock arm. Lock arm may be positioned such that it moves along an outer surface of cam 214, as a user moves release 210. FIG. 7A depicts dispensing lock 200 in a locked state (in some embodiments, a locked state is the natural resting state of dispensing lock 200). In a locked state first engager 222 may be positioned such that it engages cam tracker 216. When first engager 222 engages cam tracker 216, the cam tracker is inhibited from moving through cam track 218. Inhibition of cam tracker 216 moving through cam track 218 consequently inhibits movement of reservoir 208 and loading of any medicant into the inhaler air path. In some embodiments, first portion 204 may be configured to rotate in only one direction relative to second portion 206.

FIG. 7B depicts dispensing lock 200 in an unlocked state (in some embodiments, a locked state is the natural resting state of dispensing lock 200, and therefore a user would have to in some manner apply force to release 210 to keep the dispensing lock in an unlocked state). In an unlocked state first engager 222 may be positioned such that it does not inhibit movement of cam tracker 216. Release 210 may be moved from a first position (e.g., locked) to a second position (e.g., unlocked) which moves lock arm 220 from a first position (depicted in FIG. 7A) to a second position (depicted in FIG. 7B). When first engager 222 engages cam tracker 216, the cam tracker is inhibited from moving through cam track 218.

In some embodiments, dispensing lock 200 is inhibited from moving from a locked state to an unlocked state by spring 223. Spring 223 may be formed from any of a various known materials for forming springs (e.g., metal or plastic). Spring 223 may be configured to return to a relaxed first state (depicted in FIG. 7A) after being compressed into a second state (depicted in FIG. 7B) by an applied force (e.g., from a user) when the applied force is removed.

In some embodiments, a dispensing lock may include at least two separate “locking stages”. FIGS. 7A-B depict a first stage of a dispensing lock. FIGS. 7C-D depict a second stage of a dispensing lock. Lock arm 220 may include second engager 224 coupled to or formed as part of the lock arm. In a locked state second engager 224 may be positioned such that it engages cam tracker 216 at a point after the cam tracker has moved beyond first engager 222 (depicted in FIG. 7C). When second engager 224 engages cam tracker 216, the cam tracker is inhibited from moving through cam track 218 past a certain point during rotation of first portion 204 relative to second portion 206. Inhibition of cam tracker 216 moving through cam track 218 consequently inhibits movement of reservoir 208 and loading of any medicant into the inhaler air path.

FIG. 7D depicts dispensing lock 200 in an unlocked state (in some embodiments, a locked state is the natural resting state of dispensing lock 200, and therefore a user would have to in some manner apply force to release 210 to keep the dispensing lock in an unlocked state). In an unlocked state second engager 224 may be positioned such that it does not inhibit movement of cam tracker 216. Release 210 may be moved from a first position (e.g., locked) to a second position (e.g., unlocked) that moves lock arm 220 from a first position (depicted in FIG. 7C) to a second position (depicted in FIG. 7D). When second engager 224 engages cam tracker 216, the cam tracker is inhibited from moving through cam track 218 past a certain point in a rotation of first portion 204 relative to second portion 206. An at least second locking stage inhibits accidental unlocking of dispensing lock 200 and actuation of activation mechanism 202 by, for example, a child. A second locking stage requires that release 210 be moved from a first locked position to a second unlocked position and held there until first portion 204 has been rotated relative to second portion 206 at least a certain percentage (e.g., at least 90°) of 1 full rotation until cam tracker 216 has moved through cam track 218 past second engager 224.

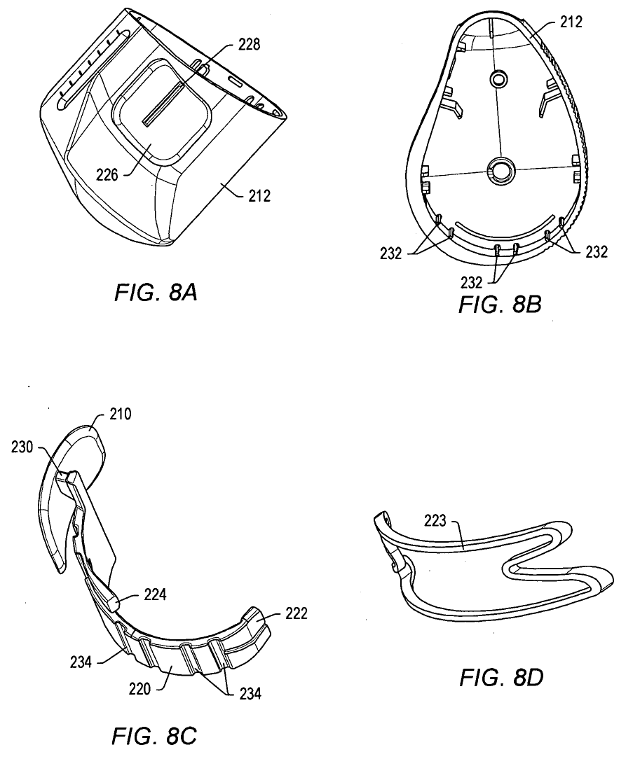

FIGS. 8A-D depict a representation of a perspective view of an embodiment of several parts used to form a dispensing lock release mechanism of an inhaler. FIGS. 8A-B depict representations of two different perspective views of an embodiment of second portion cover 212. Second portion cover 212 may include recess 226. Recess 226 provides a depression in the surface of second portion cover 212 for release 210 to fit in and move along an axis from a first locked position to a second unlocked position. In some embodiments, recess 226 may be of a depth such that release 210 remains substantially flush with the exterior surface of second portion cover 212 (e.g., to reduce its visibility to children).

Second portion cover 212 may require a greater wall thickness to accommodate recess 226, relative to a second portion cover without a recess and/or dispensing lock (a size of cap 102 (depicted in FIG. 9) may be increased to accommodate the greater wall thickness of second portion cover 212). Release 210 may be color matched to second portion cover 212 to reduce its visibility to children. Second portion cover 212 may include opening 228 positioned in recess 226. A shape of opening 228 may be configured to restrict the direction of movement of release 210. Elongated member 230 may couple release 210 to lock arm 220 through opening 228.

In some embodiments, second portion cover 212 may include two or more projections 232 (depicted in FIG. 8B). Projections 232 may function to provide support for and assist in appropriately positioning, for example, lock arm 220 and/or spring 223 within second portion cover 212. FIG. 8C depicts a detailed representation of an embodiment of main portion of dispensing lock 200, including release 210, lock arm 220, first engager 222, second engager 224, and elongated member 230. In some embodiments, lock arm 220 may include one or more recesses 234. Recesses 234 may function to inhibit dispensing lock 200 from jamming during use. FIG. 8D depicts a detailed representation of an embodiment of spring 223.

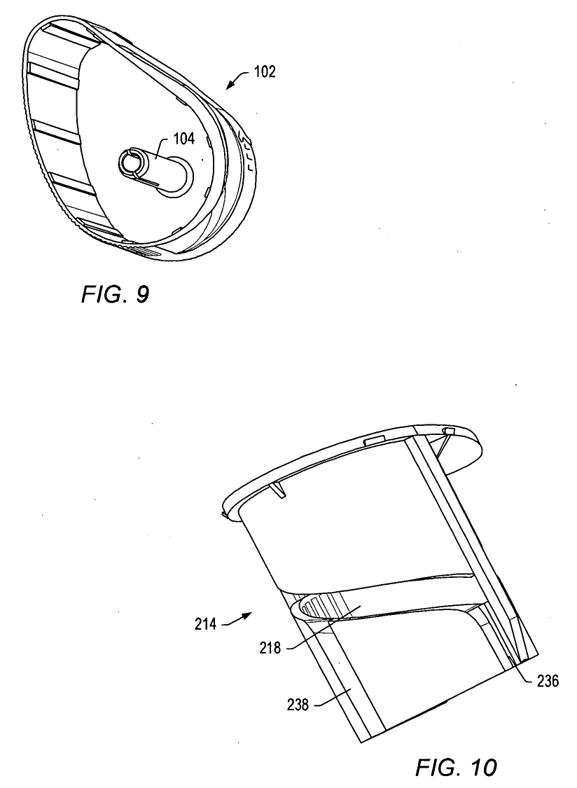

FIG. 10 depicts a detailed representation of cam 214 and cam track 218. In some embodiments, first engager 222 and/or second engager 224 may be bent inward, relative to lock arm 220, towards the exterior surface of cam 214. First engager 222 and/or second engager 224 may be bent inward to more fiercely engage cam tracker 216. Cam 214 may include first engager track 236 which functions to accommodate first engager 222 such that lock arm 220 may lay substantially flush against the exterior surface of the cam. Cam 214 may include second engager track 238 which functions to accommodate second engager 224 such that lock arm 220 may lay substantially flush against the exterior surface of the cam. A cam may include an engager track for every engager coupled to a lock arm.

In some embodiments, an inhaler may include a multiple dose inhibitor system (overdosing inhibitor) which functions to inhibit a prescribed user from activating an inhaler multiple times such that more than one dose of medicant is released.

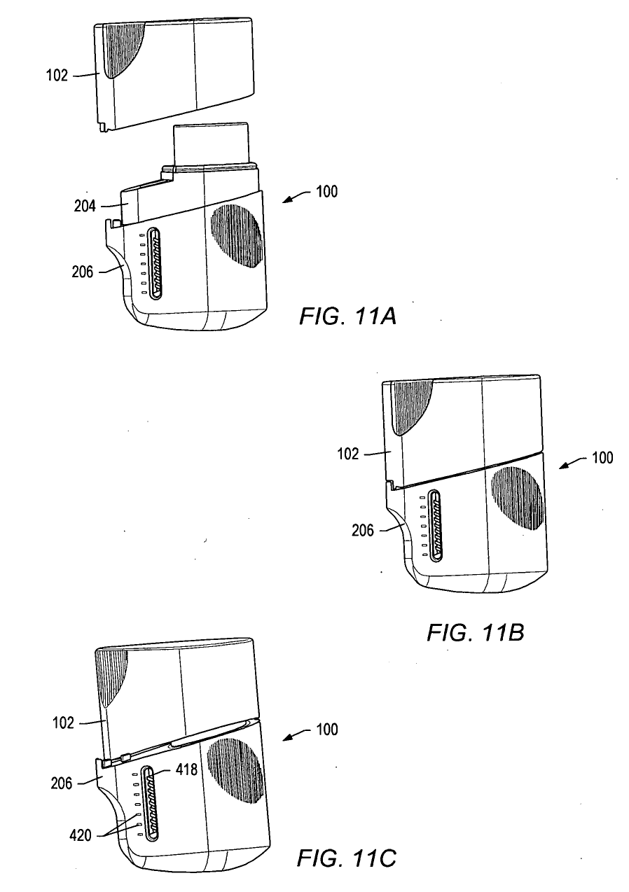

FIGS. 11-18 depict a representation of an embodiment of a multiple dose inhibitor system 300 (overdosing inhibitor 300) for an inhaler 100. Overdosing inhibitor 300 may function to inhibit actuating inhaler 100 without using cap 102. In some embodiments, overdosing inhibitor 300 may function to inhibit rotation in a first direction (e.g., counter clockwise rotation) of first portion 204 relative second portion 206, while allowing rotation in a second direction (e.g., clockwise rotation). Overdosing inhibitor 300 may function to inhibit loading of inhaler 100 with medicant without cap 102 coupled to inhaler body 110. Overdosing inhibitor 300 may inhibit actuation of the activation mechanism without cap 102.

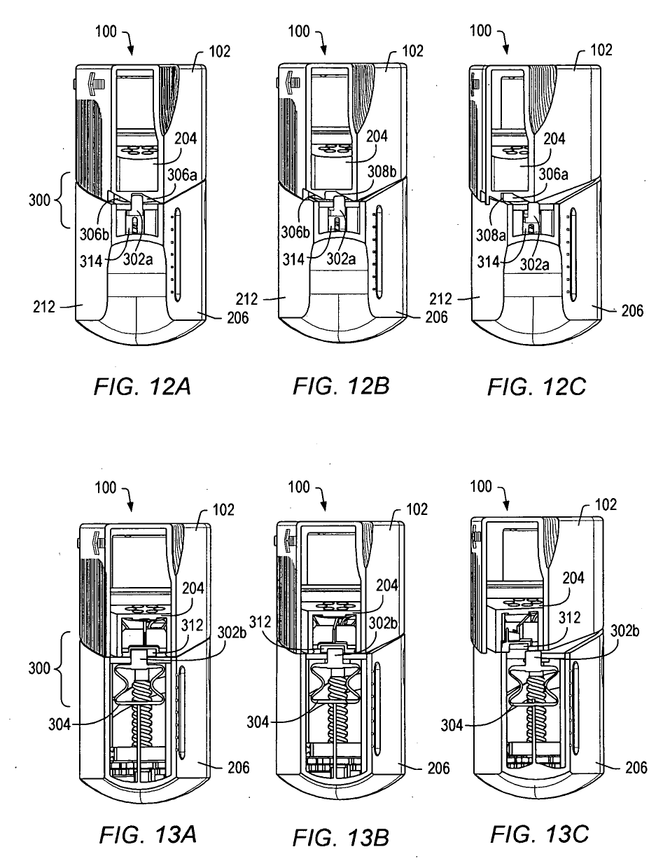

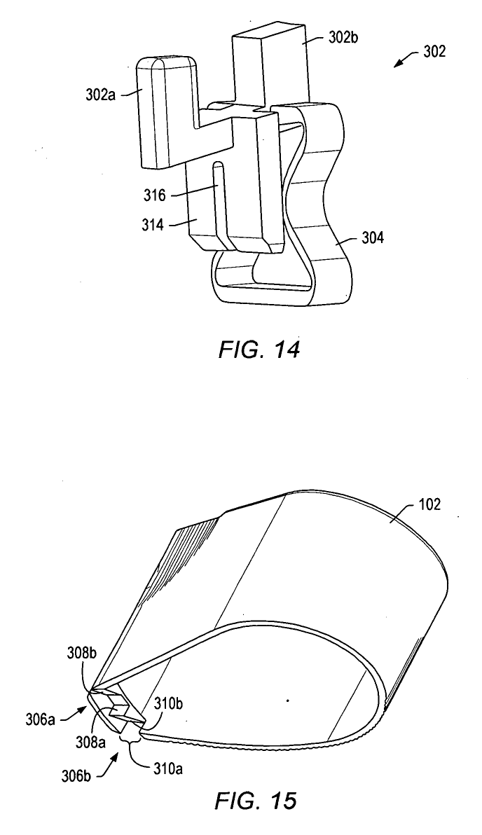

In some embodiments, overdosing inhibitor 300 may include a locking pin 302. Locking pin 302 may include pins 302a and 302b, as depicted in FIG. 14. Locking pin 302 may function to inhibit multiple actuations of activation mechanism 202. Locking pin 302 may be released by rotating cap 102 in the second direction. An embodiment of a method of using overdosing inhibitor 300 is depicted in FIGS. 11A-C. Cap 102 may be pushed into position on first portion 204 of inhaler 100, as depicted in FIG. 11A. Upon assembling inhaler 100, cap 102 may be rotated to actuate activation mechanism 202 as depicted in FIG. 11B. Rotating cap 102 may push down locking pins 302a,b as depicted in FIG. 11C and FIGS. 12A-C.

In some embodiments, overdosing inhibitor 300 may include locking pin 302. In some embodiments, locking pin 302 may be spring-loaded and include spring element 304 (e.g., as depicted in FIGS. 13-14). Spring loaded locking pin 302 may be activated by rotation of cap 102. Locking pin 302 may inhibit any rotation of reservoir 208 in any direction (only a small degree in clockwise direction is possible) with cap 102 removed.

FIGS. 12A-C depict a representation of a partial cross-sectional view of an embodiment of an overdosing inhibitor 300 for inhaler 100. In FIGS. 12A-C a sectional view is depicted which cuts away a portion of inhaler 100 displaying an interference between first pin 302a and cap 102. Cap 102 includes first pin opening 306a and second pin opening 306b. When cap 102 is initially positioned on inhaler 100 and in such a starting position first pin 302a is positioned in first pin opening 306a. First pin opening 306a includes a blocking straight sidewall 308a that runs parallel to first pin 302a, such that in the embodiment depicted in FIG. 12A cap 102 is inhibited from rotating in a counter clockwise direction. First pin opening 306a includes angled side wall 308b which runs at an angle relative to first pin 302a, such that in the embodiment depicted in FIG. 12 cap 102 may rotate in a clockwise direction (e.g., as depicted in FIGS. 12B-C). As cap 102 is rotated first pin 302a is pushed down into second portion cover 212. After cap 102 rotates past first pin 302a (e.g., occurring after about 50° of rotation), the first pin may reposition itself back into is original position extending above second portion cover 212.

After cap 102 is rotated in a clockwise direction to about 330° towards its original position, the cap will approach second pin opening 306b (See FIG. 15). Second pin opening 306b includes opening 310a in the side wall of cap 102 allowing first pin 302a to enter second pin opening 306b as cap 102 is rotated in a clockwise direction. Second pin opening 306b includes angled sidewall 310b that runs at an angle relative to first pin 302a, such that in the embodiment depicted in FIG. 15 cap 102 may rotate in a clockwise direction. As angled wall 310b is rotated with cap 102 first pin 302a is pushed down into second portion cover 212 until cap 102 rotates enough such that first pin opening 306a is positioned over first pin 302a allowing first pin 302a to position within first pin opening 306a. In some embodiments, as first pin 302a positions within first pin opening 306a, the force of the first pin moving into first pin opening 306a and striking an upper surface of the first pin opening may make a noise (e.g., a clicking noise which may notify a user that the inhaler has been activated and a dose of medicant is ready for dispersing).

In some embodiments, elongated member 104 of cap 102 may function to inhibit loading of multiple doses of medicant in dispenser opening 106 when the cap is coupled to inhaler 100. Upon actuation of inhaler 100 by rotating cap 102, a dose of medicant is loaded into dispenser opening 106. In some embodiments, elongated member 104, when positioned in dispenser opening 106, may function to inhibit loading of more than one dose of medicant in dispenser opening 106 (e.g., no matter how many times the inhaler is actuated).

In some embodiments, second pin opening 306b may include sidewall 310b, however, the sidewall may not be angled. The sidewall may be straight running substantially parallel to first pin 302a, such that in the embodiment depicted in FIG. 12 cap 102 is inhibited from rotating in a clockwise direction beyond sidewall 310b. An advantage of such an embodiment is that after activation of an inhaler a user is required to remove cap 102 and reposition the cap to its original starting position such that first pin 302a is repositioned in first pin opening 306a. As such a user is then aware whether or not an inhaler has been activated. In some embodiments, cap 102 may not include second pin opening 306b that accomplishes a similar effect as when second pin opening 306b includes a straight sidewall 310b.

FIGS. 13A-C depict a representation of a partial cross-sectional view of an embodiment of an overdosing inhibitor 300 for inhaler 100. In FIGS. 13A-C a section view is depicted which cuts away a portion of inhaler 100 displaying an interference between second pin 302b and reservoir 208. Reservoir 208 includes pin opening 312 (e.g., depicted in FIGS. 13 and 18A-B). When cap 102 is initially positioned on inhaler 100 and in such an inactivated starting position second pin 302b is positioned in pin opening 312. In some embodiments, pin opening 312 may not extend entirely through a surface of reservoir 208 (opening 312 might be described as more of a recess) such that when an inhaler is assembled a user may not have access to or see second pin 302b.

The interference between second pin 302b and reservoir 208 when the second pin is positioned in pin opening 312 inhibits rotation of first portion 204 relative to second portion 206 of inhaler 100 and the resulting loading of a dose of medicant. FIG. 14 depicts a representation of locking pin 302 and the relationship between first pin 302a and second pin 302b such that the first and the second pin are coupled together. As cap 102 rotates depressing first pin 302a, second pin 302b is also depressed out of pin opening 312 and below reservoir 208 such that inhaler 100 is unlocked and reservoir 208 may be rotated in order to prepare a dose of medicant.

Spring element 304 may be formed from any of various known materials for forming springs (e.g., metal or plastic). Spring element 304 may be configured to return to a relaxed first locked state (depicted in FIGS. 13A and 14) after being compressed into a second unlocked state (depicted in FIG. 13C) by an applied force (e.g., from a user rotating cap 102) when the applied force is removed.

In some embodiments, locking pin 302 may include guide element 314. Guide 314 may include guide channel 316. Guide 314 may be positioned in guide opening 318 of second portion cover 212 (e.g., depicted in FIG. 16). Guide opening 318 may inhibit lateral movement of guide 314 (and therefore locking pin 302) once the guide is positioned in the guide opening. Extension 320 of second portion cover 212 may function to support spring element 304 from below during compression of the spring element. As spring element 304 is compressed into an unlocked state, guide channel 316 moves vertically with the spring element such that the guide channel slides over extension 320 further inhibiting lateral movement of locking pin 302 while allowing spring element 304 to compress without interference occurring between extension 320 and guide 314.

In some embodiments, inhaler may include one or more interference inhibitors 322. Interference inhibitor 322 may function to prevent movement of first locking pin 302a without using cap 102, for example preventing accidental movement of locking pin 302a and subsequent accidental actuation of inhaler 100. FIGS. 16-17 depict an embodiment of interference inhibitor 322. First portion cover 324 may fit over a proximal end of reservoir 208 covering second locking pin 302b. Outer dimensions of inhaler 100 may be extended in the area of the locking pin 302 to accommodate overdosing inhibitor system 300, such that access to dispenser opening 106 is not inhibited.

Overdosing inhibitor 300 may be formed from any appropriate materials known to one skilled in the art. In some embodiments, overdosing inhibitor 300 may be formed from acrylonitrile butadiene styrene (ABS) plastic. Locking pin 302 may be color matched to first portion cover 324 such that the locking pin is difficult to see and therefore overdosing inhibitor 300 is more difficult to circumvent.

In some embodiments, an inhaler may include controlled dispenser system 400 (controlled dispenser 400) which functions to inhibit a prescribed user from activating inhaler 100 more than a predetermined number of times. Inhaler 100 is actuated by rotation of first portion 204 relative to second portion 206 by, for example, 360°. This action loads a dose of medicant into the air path, and the medicant is then available for inhalation. Actuation can be made in current designs countless times with the exception that medicant runs out currently after approximately, for example, 35 doses. Controlled dispenser 400 may function to inhibit a user from actuating inhaler 100 more than a prescribed number of times.

In some embodiments, a method of using inhaler 100 may include placing cap 102 into position on first portion 204 of inhaler 100. Upon assembling inhaler 100, cap 102 may be rotated to actuate the activation mechanism. After actuation of inhaler 100, cap 102 may be removed and a user may then inhale a dose of medicant from dispenser opening 106. After dispensing of a dose of medicant, cap 102 may be replaced onto inhaler 100. Controlled dispenser 400 functions to inhibit rotation of the reservoir assembly against the body after the end of inhaler life. A mechanism itself may be invisible to users and automatically locks the inhaler after a predetermined number of activations (e.g., a 30^{th} dose). Controlled dispenser 400 does not interfere with actuation of inhaler 100 until the inhaler has been actuated a predetermined number of times.

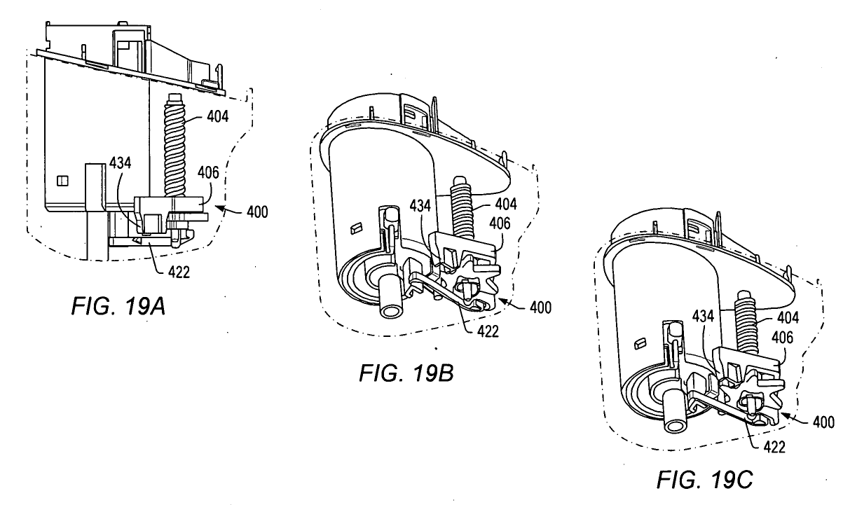

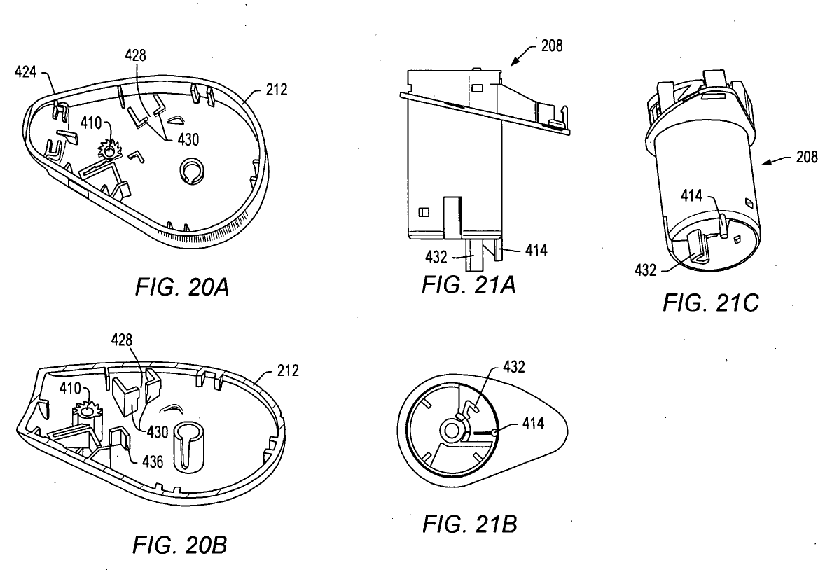

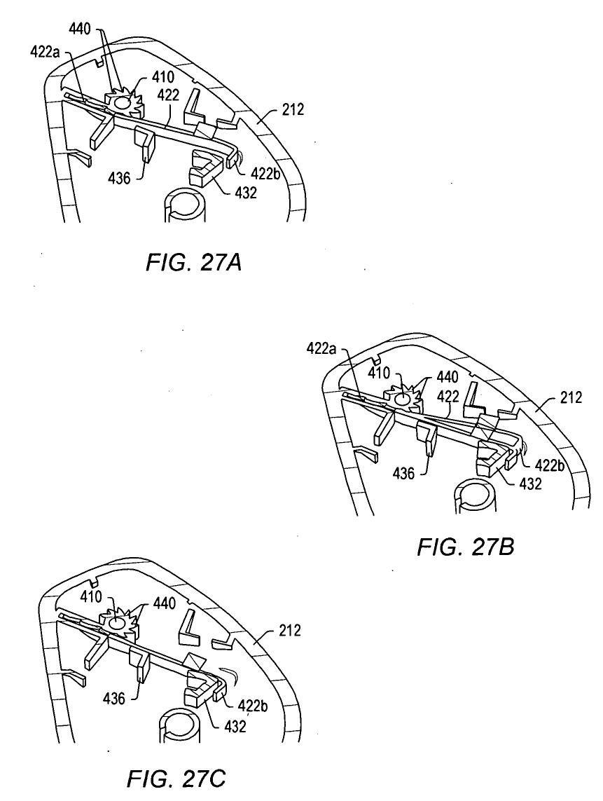

FIGS. 19-28 depict a representation of an embodiment of a controlled dispenser system 400 for inhaler 100. Controlled dispenser system 400 is incorporated into the dose counter system 402. The dose counter system may include threaded counter 404 and indicator 406. The proximal end of counter 404 may fit in opening 408 in cam 214 (e.g., depicted in FIG. 23), while the distal end of the counter may fit in opening 410 of second portion cover 212 (e.g., depicted in FIGS. 20 and 27) inhibiting movement of the counter while allowing the counter to still rotate. Counter 404 may include sprocket 412 positioned at the distal end of the counter. Indicator 406 may be threaded on counter 404.

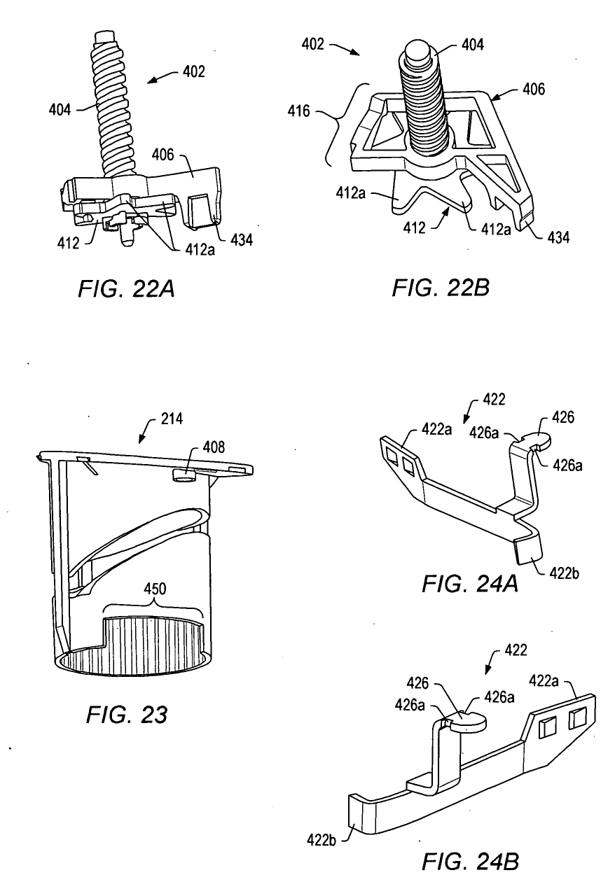

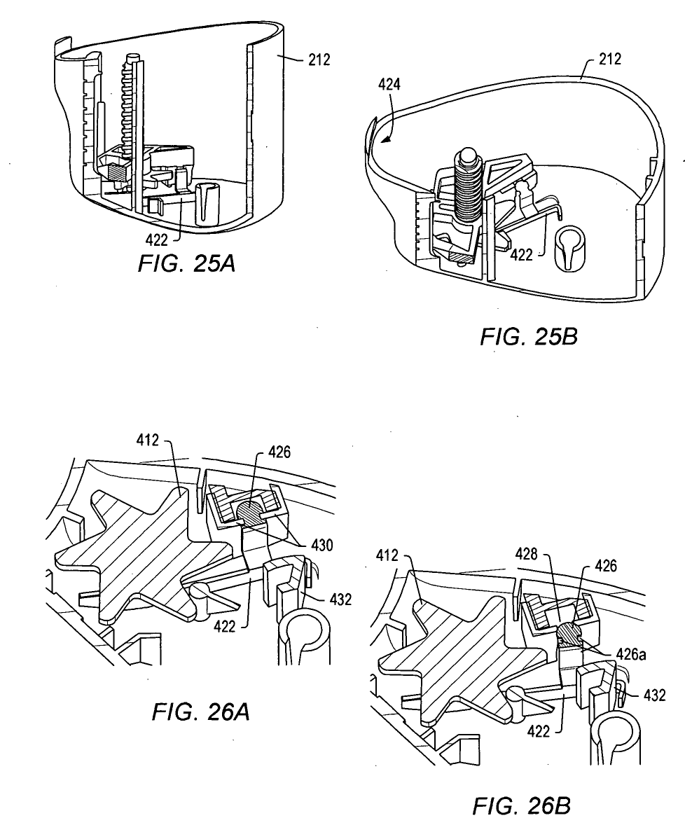

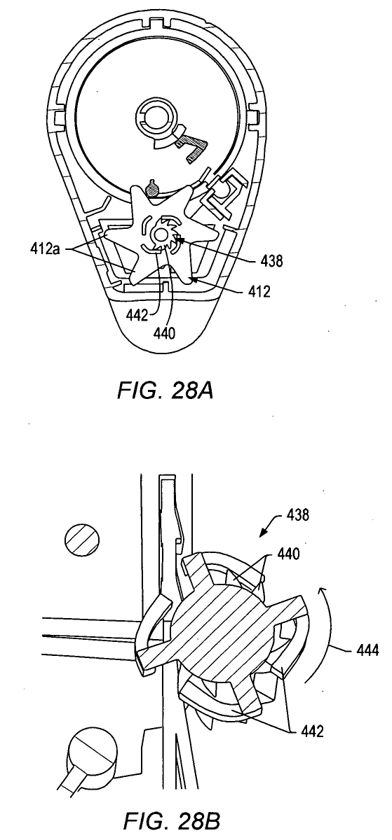

Reservoir 208 may include member 414 coupled to the distal end of the reservoir (e.g., as depicted in FIG. 21A-C). As reservoir 208 rotates during actuation of inhaler 100, member 414 also rotates with the reservoir such that the member engages sprocket 412. As member 414 engages sprocket 412, the sprocket rotates counter 404. Sprocket 412 may include teeth 412a. Teeth 412a may be oriented in a predetermined direction of rotation (e.g., as depicted in FIG. 28A) to better engage member 414. Rotation of sprocket 412 moves threaded indicator 406 down counter 404 (e.g., as depicted in FIGS. 25-26). Portion 416 of indicator 406 (e.g., as depicted in FIG. 22B) may have a shape complementary to front 424 of second portion cover 212. Portion 416 of indicator 406 may be positioned in front 424 of second portion cover 212 such that indicator 406 is inhibited from rotating with counter 404 as it rotates forcing indicator 406 to move vertically along counter 404 as it rotates (e.g., as depicted in FIGS. 25A-B).

Every time first portion 204 is rotated relative to second portion 206 to actuate inhaler 100, indicator 406 moves vertically along counter 404. Movement of indicator 406 along counter 404 may be viewed by a user through opening 418 (e.g., as depicted in FIG. 11C). In some embodiments, opening 418 may include a transparent cover to protect internal systems in inhaler 100. As indicator 406 moves along counter 404, indicia 420 positioned along opening 418 may indicate to a user how many doses of medicant have been dispensed and/or how many doses of available medicant remain in inhaler 100.

In some embodiments, controlled dispenser system 400 may include a spring lock 422 (e.g., as depicted in FIGS. 24A-B). Spring lock 422 may include first end 422a. First end 422a of spring lock 422 may function to couple the spring lock to second portion cover 212 (e.g., as depicted in FIGS. 27A-C). Spring lock 422 may include second end 422b. Second end 422b may function to inhibit rotation of reservoir 208 when the second end is moved from a first unlocked position (e.g., as depicted in FIG. 27A) to a second locked position (e.g., as depicted in FIG. 27C) and hence inhibit further activation of inhaler 100. The first unlocked position of second end 422b may include an activated state of spring lock 422 containing potential energy. Movement of spring lock 422 from first unlocked position to second locked position releases the potential energy of spring lock 422.

In some embodiments, spring lock 422 may include tab 426. Tab 426 may function to couple spring lock 422 to second portion cover 212 via slot 428. Walls 430 defining slot 428 may engage channels 426a of tab 426 holding spring lock 422 in an unlocked position.

Reservoir 208 may include second member 432 (e.g., as depicted in FIGS. 21 and 26-27). Second member 432 may extend from the distal end of reservoir 208. When spring lock 422 is released from an unlocked position to a locked position second end 422b of the spring lock may engage member 432 of reservoir 208. When spring lock 422 engages member 432 of reservoir 208, the reservoir is inhibited from rotating and therefore inhaler 100 is inhibited from actuating again.

Indicator 406 may include release 434. Release 434 may extend towards a distal end of inhaler 100 (e.g., as depicted in FIG. 22). As inhaler 100 is actuated and dispenses medication, indicator 406 moves vertically downward along counter 404 with each dose of dispensed medicant. After a predetermined (e.g., 30 doses of medicant) number of doses of medicant have been dispensed, release 434 may reach a distal end of an interior of second portion cover 212. As release 434 reaches a distal end of second portion cover 212, the release may position itself in slot 428 (e.g., as depicted in FIG. 26A).

Release 434 may include a cross-sectional shape such that as the release enters slot 428, the release does not engage or interfere with tab 426. Release 434 may taper towards a distal end of the release such that the distal end fits within slot 428. However, a width of release 434 may increase going up from the distal end of the release towards a proximal end of the release. Toward the proximal end of release 434, the width of the release may be greater then a width of slot 428. Tapered release 434 thus effectively functions as a wedge as the release enters slot 428, eventually the release spreads walls 430 of slot 428 out enough that the walls disengage from channels 426a of tab 426 (e.g., as depicted in FIG. 26B) releasing spring lock 422. Released spring lock 422 engages member 432 inhibiting the actuation of inhaler 100 further. FIGS. 27A-C depict a representation of a cross-section of spring lock 422 moving from a first unlocked position to a second locked position, the cross-section depicted is seen at a level below that of tab 426.

In some embodiments, second portion cover 212 may include limit stop 436 positioned at a distal end on an interior surface of the second portion cover (e.g., as depicted in FIGS. 27A-C). Limit stop 436 may function to inhibit second end 422b of released spring lock 422 from moving beyond a point of possible engagement with member 432, if member 432 has not yet rotated into a position to which second end 422b may engage the member.

In some embodiments, cam 214 may include opening 450 (e.g., as depicted in FIG. 23). Opening 450 may function to allow second end 422b of released spring lock 422 to pass beyond cam 214 such that the second end may engage member 432 of reservoir 208.

In some embodiments, an inhaler may include a system that inhibits first portion 204 from rotating in a first direction while allowing rotation in a second direction relative to second portion 206 of inhaler 100. In some embodiments, directional rotation control system may include ratchet system 438. Ratchet system 438 may include teeth 440 (e.g., as depicted in FIGS. 27A-C and 28A-B) positioned on an exterior surface surrounding opening 410 of second portion cover 212. Teeth 440 may be directed outward and curve in a direction of allowed rotation 444 between first portion 204 and second portion 206 of inhaler 100. Counter 404 may include one or more ratchet members 442 coupled to a distal end of the counter below sprocket 412. Ratchet members 442 may be positioned such that they engage teeth 440 (e.g., as depicted in FIGS. 28A-B). Ratchet members 442 may curve in a direction of inhibited rotation between first portion 204 and second portion 206 of inhaler 100. Ratchet members 442 may engage teeth 440 slipping over the curved surface of the teeth with little effect while rotating in a direction of allowed rotation 444 (e.g., as depicted in FIG. 28B). However, when a user attempts to rotate first portion 204 relative to second portion 206 in a direction opposite to allowed rotation 444, distal end 446 of ratchet members 442 may actively engage flat surface 448 of teeth 440 inhibiting rotation in the direction opposite to rotation 444.

In some embodiments, an inhaler may include a security fitting which functions to inhibit a child and/or a drug addict from accessing residual medicant in an inhaler once a user has finished using the inhaler. In some embodiments, such a security fitting may be used in combination with any other child resistant features and/or security features described herein and/or elsewhere.

In some embodiments, a security fitting may function, after application, to prevent access to unused medicant in an inhaler when the inhaler is full (e.g., the inhaler has never been used). A security fitting, after application, may function to prevent access to unused medicant in an inhaler when the inhaler is actuated multiple times. A security fitting, after application, may function to prevent access to unused medicant in an inhaler when the inhaler is shaken and/or manipulated in different orientations. In some embodiments, a security fitting may be configured such that after insertion the security fitting may not be removed.

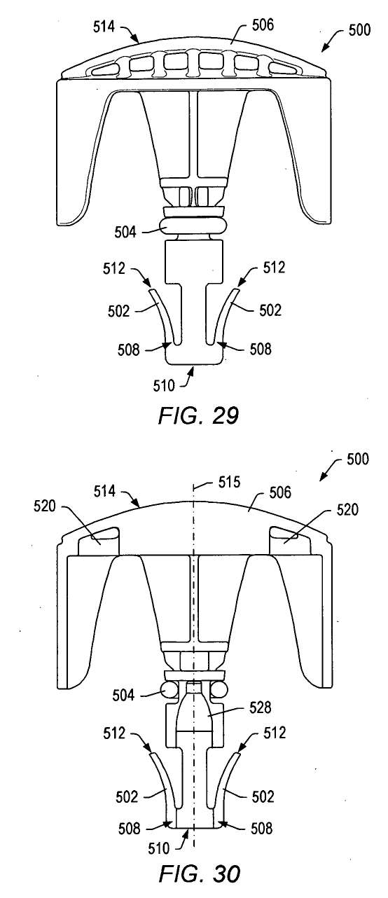

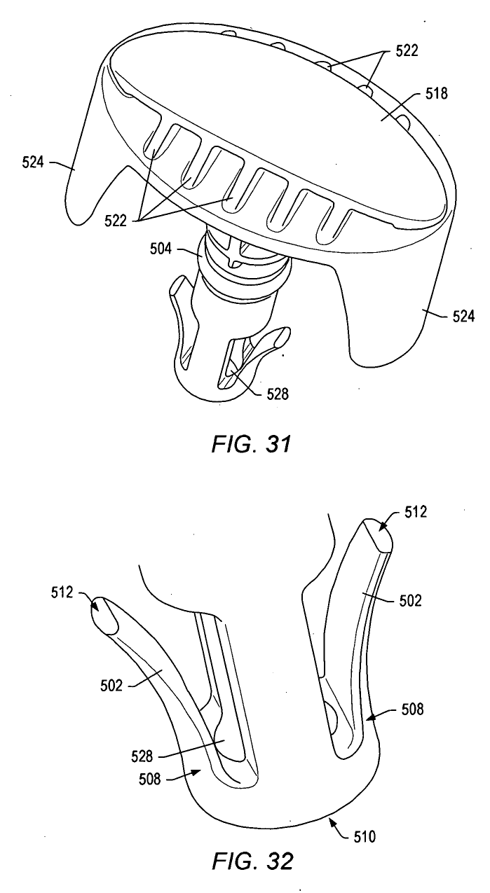

FIGS. 29-38 depict representations of embodiments of a security fitting 500 for inhaler 100. In some embodiments, security fitting 500 may include retention members 502, sealing member 504, and cover 506. A distal end of security fitting 500 may function to be positioned in dispenser opening 106. Security fitting 500 may function to seal dispenser opening 106 to inhibit access to medicant in reservoir 208 or any residual medicant in the dispenser opening. Security fitting 500 may be shaped such that portions of the security fitting have a complementary shape to dispenser opening 106. Portions of security fitting 500 having a complementary shape and/or size to dispenser opening 106 may inhibit movement of the security fitting relative to inhaler 100 once the security fitting is positioned in the dispenser opening to prevent twisting and lateral forces from damaging the retention members 502.

Inhibiting movement of a positioned security fitting relative to the inhaler may function to prevent removal of the activated security fitting. A person trying to remove a positioned security fitting is able to gain less leverage on the security fitting when the security fitting is positioned tightly in the dispenser opening of the inhaler.

In some embodiments, a distal end of security fitting 500 may include retention members 502 coupled to the distal end of the security fitting. Security fitting 500 may include two retention members 502 coupled to the distal end of the security fitting. In some embodiments, a security fitting may include one, two, three, four, or more retention members. Retention members 502 may be slightly flexible such that when a force is applied to the retention members they may deform from a first shape into a second shape. However, upon cessation of the applied force the retention members may reform back into the first shape.

Distal end 508 of retention member 502 may be coupled to distal end 510 of security fitting 500 (e.g., as depicted in FIGS. 29-30). Proximal end 512 of retention member 502 may extend away from distal end 510 of security fitting 500 towards proximal end 514 of security fitting 500. Proximal end 512 of retention member 502 may be directed away from central longitudinal axis 515 of security fitting 500 (e.g., as depicted in FIGS. 29-30). Proximal end 512 of retention member 502 may flex towards security fitting 500 when force is applied to the proximal ends. When flexed inwards proximal ends may fall within an outer diameter of sealing member 504 (e.g., during the process of positioning security fitting 500 in the dispenser opening). In a natural inflexed state proximal end 512 of retention member 502 may extend out and away from security fitting 500 and fall outside of an outer diameter of sealing member 504 (e.g., after security fitting 500 has been positioned). Upon positioning security fitting 500 in inhaler 100 retention members 502 may function to inhibit removal of security fitting 500.

Retention members 502 may include a profile that minimizes strain and allows retention members to fold in during insertion without causing peaks of strain. Profile of retention members 502 may function to cause strain to be equally distributed along their length during insertion of security fitting 500 into inhaler 100, minimizing the risk of damage to the extension members. In some embodiments, distributing strain equally may be accomplished by the cross-section of the arms becoming smaller towards the arm tip, and the curve of the arm also reducing towards the tip.

In some embodiments, distal end 510 may include opening 528 (e.g., as depicted in FIG. 30). Opening 528 may accommodate proximal end 530 of dose pin 532 (e.g., as depicted in FIGS. 35-36). Once security fitting 500 is positioned the security fitting may inhibit inhalation of medicant from inhaler 100.

In some embodiments, security fitting 500 may inhibit the coupling of cap 102 to inhaler 100. In some embodiments, security fitting 500 may be combined with overdosing inhibitor 300, such that once the security fitting is positioned, the security fitting may inhibit cap 102 from being positioned. If cap 102 is inhibited from being positioned, overdosing inhibitor 300 subsequently inhibits actuation of inhaler 100.

In some embodiments, security fitting 500 may include sealing member 504. Sealing member 504 may function to form a seal between security fitting 500 and dispenser opening 106 inhibiting access to medicant in inhaler 100. Security fitting 500 may include a seal recess 516. Note that a distal lower radius in seal recess 516 is larger than proximal upper radius, to assist in the prevention of the removal of the plug by force. Seal recess may function as a reduced outer diameter channel (relative to the diameter of a distal end of dispenser opening 106) wherein sealing member 504 may be seated, inhibiting movement of the sealing member.

In some embodiments, sealing member 504 may include a relatively large cross-section offering the ability to absorb tolerance variations in the molded portions of the security fitting. The sealing member may be formed from butyl, EPDM, and/or silicone.

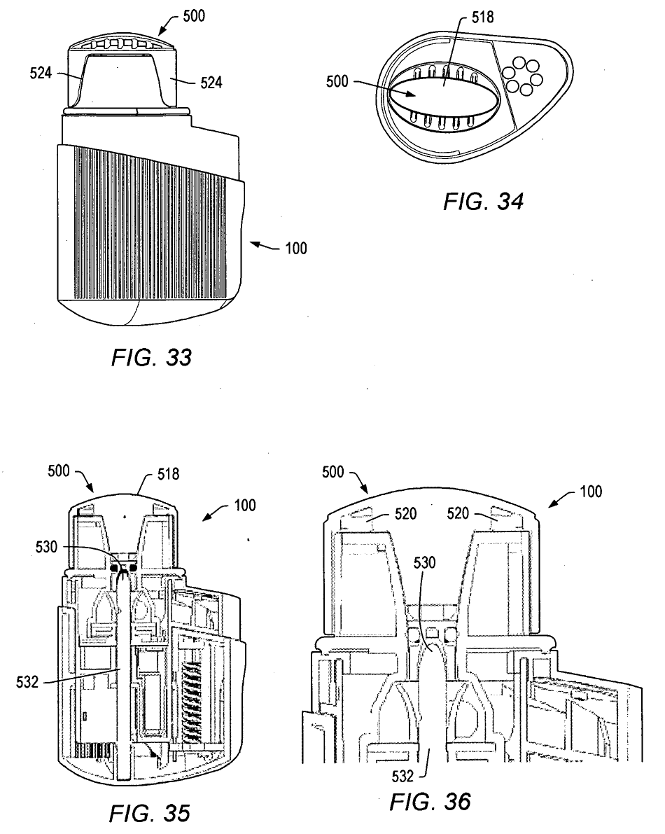

In some embodiments, security fitting 500 may include cover 506. Cover 506 may function to fit over dispenser opening 106 once security fitting 500 is positioned (e.g., as depicted in FIGS. 33-36). Cover 506 may include impact area 518, fluid path 520, extensions 522, and seal inhibitors 524.

Impact area 518 may function as a point wherein a user may more easily apply pressure to security fitting 500 in order to position the security fitting in dispenser opening 106. A certain amount of force is necessary to apply to the security fitting in order to move sealing member 504 into dispenser opening 106. Impact area 518 may include a substantially large smooth space increasing contact area between a user's hand (e.g., palm) and impact area 518.

In some embodiments, cover 506 may include fluid path 520. Fluid path 520 may function to inhibit the risk of a user choking on a security fitting 500. A risk of choking may exist, for example, in the event that a user may partially assemble and/or incorrectly assemble the security fitting and the inhaler and then tried to inhale on it, causing it to become detached from the inhaler and stick in a user's throat. Fluid paths 520 may function to allow fluids (e.g., air, saliva) to pass beyond security fitting 500 if positioned a child's or a user's throat. In some embodiments, portions of security fitting 500 may be configured to comply with BS 7272: 2000 Part 1 (Writing and marking instruments. Specification for safety caps); BS 7272: 2000 Part 2 (Writing and marking instruments. Specification for end closures); and/or BS EN 71-1:2005 (Safety of toys. Mechanical and physical properties).

In some embodiments, cover 506 may include two or more extensions 522. Extensions 522 may function to inhibit a user or child from plugging fluid path 520 if, for example, security fitting 500 is swallowed. Cover 506 may include two extensions 502, three extensions 502, four extensions 502, five extensions 502, or six extensions 502 on either side of impact area 518.

In some embodiments, cover 506 may include seal inhibitors 524. Cover 506 may include two or more seal inhibitors 524 (e.g., as depicted in FIGS. 29-31). Seal inhibitors 524 may function to inhibit a seal being formed around cover 506 by a user's lips. FIGS. 33-36 depict a representation of an embodiment of cover 506 positioned in inhaler 100. Seal inhibitors 524 may function to disrupt the smooth surface of first portion 204 such that the mouth of a human is inhibited from forming a seal around first portion 204.

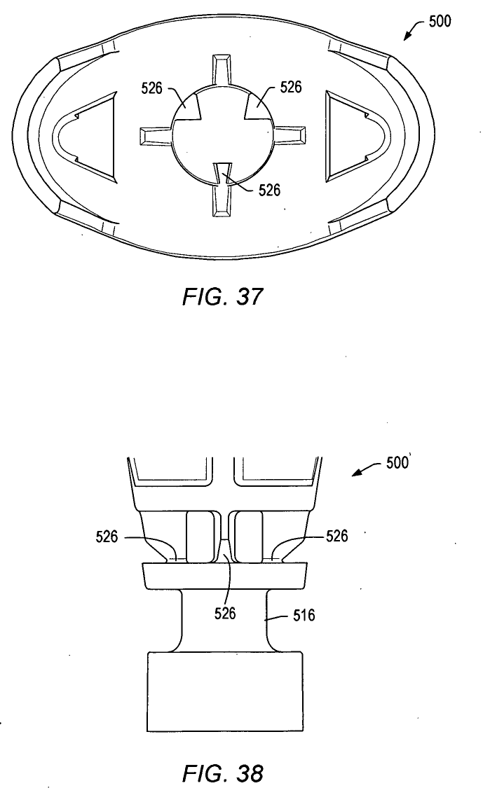

In some embodiments, security fitting 500 may include frangible portion 526. Frangible portion 526 may function to break if excessive removal force is applied to security fitting 500 after insertion to inhaler 100. Upon breaking of frangible portion 526, distal end 510 of security fitting 500 and sealing member 504 may remain in dispenser opening 106, while cover 506 may be removed from the dispenser opening. Frangible portion 526 is depicted in FIGS. 37-38.

Security fitting 500 may be formed from any number of materials known to one skilled in the art. Security fitting may be formed from a tough high-flow engineering polymer with good creep resistance (e.g., Dupont Delrin 900P).

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1-3. (canceled)

4. An inhaler, comprising:
an inhaler body comprising a first portion and a second portion, wherein the first portion comprises a dispenser opening configured to dispense a dose of medicant upon activation of the inhaler body, and wherein the inhaler body is activated by rotation of the first portion relative to the second portion; and
a dispensing lock configured to inhibit rotation of the first portion relative to the second portion of the inhaler when the dispensing lock is in a first locked position, wherein when the dispensing lock is in a second unlocked position rotation of the first portion relative to the second portion of the inhaler is allowed.

5-9. (canceled)

10. An inhaler, comprising:
an inhaler body comprising a first portion and a second portion, wherein the first portion comprises a dispenser opening configured to dispense a dose of medicant upon activation of the inhaler body, and wherein the inhaler body is activated by rotation of the first portion relative to the second portion; and
a cap configured to couple to the first portion of the inhaler body, and wherein the cap is further configured to inhibit access to the dispenser opening when coupled to the first portion of the inhaler body, wherein the cap comprises an elongated member positionable in the dispenser opening when the cap is coupled to the inhaler body, and wherein the elongated member is configured to inhibit loading of more than one dose of medicant when the elongated member is positioned in the dispenser opening; and
a multiple dose inhibitor system configured to inhibit activation of the inhaler body when the cap is uncoupled from the inhaler body.

11-36. (canceled)

37. An inhaler, comprising:
an inhaler body comprising a first portion and a second portion, wherein the first portion comprises a dispenser opening configured to dispense a dose of medicant upon activation of the inhaler body, and wherein the inhaler body is activated by rotation of the first portion relative to the second portion, wherein two or more recesses are positioned on opposing sides of an exterior surface of the first portion of the inhaler body;
a cap comprising two or more tabs positionable in two or more of the recesses such that when the tabs are positioned in the recesses the cap is coupled to the first portion of the inhaler body such that children are inhibited from accessing the dispenser opening;
a dispensing lock configured to inhibit rotation of the first portion relative to the second portion of the inhaler when the dispensing lock is in a first locked position, wherein when the dispensing lock is in a second unlocked position rotation of the first portion relative to the second portion of the inhaler is allowed;
a cap configured to couple to the proximal end of the inhaler body, and wherein the cap is further configured to inhibit access to the dispenser opening when coupled to the proximal end of the inhaler body, wherein the cap comprises an elongated member positionable in the dispenser opening when the cap is coupled to the inhaler body, and wherein the elongated member is configured to inhibit loading of more than one dose of medicant when the elongated member is positioned in the dispenser opening;
a multiple dose inhibitor system configured to inhibit activation of the inhaler body when the cap is uncoupled to the inhaler body;
a dose limitation system configured to inhibit activation of the inhaler body after the inhaler body has been activated a predetermined number of times; and
a security fitting positionable in the dispenser opening, wherein the security fitting is configured to inhibit access to medicant positioned in the inhaler body when the security fitting is positioned in the dispenser opening.

38. (canceled)

39. The inhaler of claim 37, further comprising a release configured to allow a user to move the dispensing lock from the first locked position to the second unlocked position.

40. (canceled)

41. The inhaler of claim 37, further comprising:
a second portion cover configured to inhibit access to the contents of the second portion of the inhaler body;
a cam positioned in the second portion cover, wherein the cam comprises a cam track comprising a channel substantially circling the cam; and
a reservoir positioned in the cam, wherein the reservoir is configured to contain two or more doses of medicant, and wherein the reservoir comprises a cam tracker which extends in the cam track such that as the reservoir is rotated relative to the cam, the cam tracker moves through the cam track;
wherein when the dispensing lock is in the first locked position a lock arm of the dispensing lock engages the cam tracker inhibiting the reservoir from rotating and activating the inhaler body.

42. The inhaler of claim 41, wherein the lock arm comprises:
a first engager configured to engage the cam tracker, when the dispensing lock is in a first locked position, at a first position inhibiting the reservoir from rotating beyond the first position and activating the inhaler body; and
a second engager configured to engage the cam tracker, when the dispensing lock is in a first locked position, at a second position inhibiting the reservoir from rotating beyond the second position and activating the inhaler body.

43. The inhaler of claim 37, wherein the multiple dose inhibitor system comprises a locking pin configured to inhibit rotation of the first portion relative to the second portion when in a first locked position.

44. The inhaler of claim 43, wherein the cap, when coupled to the first portion, moves the locking pin from the first locked position to second unlocked position when the first portion is rotated relative to the second portion.

45. (canceled)

46. The system inhaler of claim 37, further comprising:
a second portion cover configured to inhibit access to the contents of the second portion of the inhaler body;
a cam positioned in the second portion cover;
a reservoir positioned in the cam, wherein the reservoir is configured to contain two or more doses of medicant, and wherein the cam comprises an elongated member coupled to a distal end of the cam; and
a dose counter comprising a threaded counter coupled to the cam and the second portion cover, wherein the dose counter comprises a sprocket coupled to the distal end of the threaded counter, and wherein the dose counter comprises an indicator coupled to the threaded counter via a threaded opening in the indicator;
wherein when the first portion is rotated the reservoir is rotated a minimum degree such that the elongated member engages the sprocket rotating the threaded counter, and wherein when the threaded counter rotates the indicator moves vertically along the threaded counter towards the distal end of the second portion cover.

47. The inhaler of claim 46, wherein when the indicator moves a predetermined distance a release coupled to the indicator engages a spring lock coupled to the distal end of the second portion cover, and wherein when the released spring lock engages a second elongated member coupled to a distal end of the reservoir such that the reservoir and the first portion are inhibited from rotating relative to the second portion.

48. The inhaler of claim 47, wherein the security fitting is configured to inhibit a cap from coupling to the inhaler body when the security fitting is positioned in the dispenser opening.

49. The inhaler of claim 47, wherein the security fitting comprises a frangible portion configured to break upon application of excessive removal force to the security fitting once positioned in the dispenser opening.

50. (canceled)

51. The inhaler of claim 47, wherein the security fitting comprises a cover positioned at the proximal end of the security fitting, and wherein the cover is configured to fit over the dispenser opening.

52. The inhaler of claim 47, wherein the security fitting comprises a cover positioned at the proximal end of the security fitting, and wherein the cover is configured to fit over the dispenser opening and inhibit a cap from coupling to the inhaler body when the security fitting is positioned in the dispenser opening.

53. The inhaler of claim 47, wherein the security fitting comprises a cover positioned at the proximal end of the security fitting, and wherein the cover comprises one or more fluid paths configured to allow fluids to move through the fluid paths.

54. (canceled)

55. (canceled)

56. The inhaler of claim 47, wherein the security fitting comprises a cover positioned at the proximal end of the security fitting, and wherein the cover comprises one or more seal inhibitors configured to inhibit a seal being formed around the cover by a users lips when the security fitting is positioned in the dispenser opening.

57. (canceled)

58. The inhaler of claim 47, wherein the security fitting comprises a sealing member configured to inhibit removal of medicant from the inhaler.

59. (canceled)

60. The inhaler of claim 47, wherein the security fitting comprises one or more retention members configured to inhibit the removal of the security fitting once positioned.

61. The inhaler of claim 47, wherein the security fitting comprises one or more retention members configured to inhibit the removal of the security fitting once positioned, wherein a distal end of one or more of the retention members is coupled to a distal end of the security fitting, wherein a proximal end of one or more of the retention members, when in a first position, extends out and away from the security fitting.

62. (canceled)

63. (canceled)
